(19) United States
(12) Patent Application Publication
(10) Pub. No.: US 2023/0211133 A1
(43) Pub. Date: Jul. 6, 2023

(54) Dual Stopcock Apparatus

(71) Applicants: Antonio Joseph Chamoun, Downingtown, PA (US); Dmitry Kolesnikovich, Philadelphia, PA (US)

(72) Inventors: Antonio Joseph Chamoun, Downingtown, PA (US); Dmitry Kolesnikovich, Philadelphia, PA (US)

(21) Appl. No.: 17/569,615

(22) Filed: Jan. 6, 2022

(51) Int. Cl. A61M 25/10

(52) U.S. Cl. CPC A61M 25/10185; A61M 25/104

(57) ABSTRACT

The present disclosure provides a novel medical valve apparatus based on the three-way stopcock mechanism, but with dual inlets and side ports connected to a shared inlet and controlled by independent luer valve control mechanisms. This design enables a single operator to simultaneously inflate two separate angioplasty balloons without the need to consider pressure equalization between the balloons during bifurcated lesion procedures. Furthermore, due to the independent nature of the valve controls, the valve apparatus can equally well be used in single lesion procedures.

### FIELD OF INVENTION

The present invention relates generally to the field of medical apparatus. More specifically, the present invention relates to a novel dual stopcock design for use in cardiac operations.

### BACKGROUND

The three-way stopcock is a well-known valve design that is used for fluid flow control in conjunction with various medical apparatus, particularly in catheterization procedures. It has a main inlet (female), a main outlet (male), and a side port, and provides controlled 3-way flow from inlet to outlet, inlet to side-port or side port to outlet, by use of a handle on the top of the stopcock to open and close lines via an internal luer valve mechanism. The three-way stopcock is particularly useful in coronary angioplasty, also called percutaneous coronary intervention, which is a procedure used to open clogged heart arteries.

Angioplasty uses a tiny balloon catheter that is inserted into a blocked blood vessel to help widen it and improve blood flow to the heart. Angioplasty is often combined with the placement of a small wire mesh tube called a stent. The stent helps prop the artery open, decreasing its chance of narrowing again. Angioplasty can improve symptoms of blocked arteries, such as chest pain and shortness of breath. Angioplasty is also often used during a heart attack to quickly open a blocked artery and reduce the amount of damage to the heart.

During a cardiac angioplasty procedure an operating doctor will use medical imaging, typically live X-rays, to guide the balloon-tipped catheter across the blockage. The balloon-tipped catheter attaches to an indeflator, an inflation/deflation device used to inflate or deflate the angioplasty balloon.

The indeflator has an inflation syringe pre-filled with saline/contrast solution that is connected to the female inlet of a three-way stopcock. The balloon (and optionally a stent) is then connected to the male outlet of the stopcock. The inflation syringe has a lock to maintain negative pressure in the balloon while introducing the balloon into the vessel and positioning across the lesion. The indeflator also comprises a connecting tube coupled to the syringe, and a pressure gauge. The pressure gauge monitors the pressure developed within the angioplasty balloon while the balloon is being inflated with saline/contrast solution for maintaining the safety of inflations. Each balloon has a rated burst pressure and going above the rated burst pressure increases the risk of balloon rupture.

For single lesions where a patient has a blockage in only one artery, the aforementioned apparatus is sufficient and performs well. However, it is possible for a patient to have blockages in multiple arteries, especially in bifurcations of two adjoined vessels.

A bifurcation lesion is a coronary artery narrowing that occurs adjacent to, and/or which involves the origin of a significant side branch of the vessel. When this occurs, multiple operators and indeflators with separate balloons are required to perform a synchronized inflation of separate angioplasty balloons in the vessel and side branch in what is called a “kissing balloon technique”. It is a difficult technique, requiring careful equalization of balloon pressure across the two vessels. Poor synchronization can potentially result in unfavorable stent expansion, stent deformation, poor approximation of the stent against the vessel wall, and a potential decrease in luminal area.

Bifurcation lesions are one of the most frequently approached complex coronary lesions in everyday interventional practice.

Improved medical apparatus is required to overcome the difficulties associated with bifurcated lesions in coronary angioplasty procedures. It is within this context that the present invention is provided.

### SUMMARY

The present disclosure provides a novel medical valve apparatus based on the three-way stopcock mechanism, but with dual inlets and side ports connected to a shared inlet and controlled by independent luer valve control mechanisms. This design enables a single operator to simultaneously inflate two separate angioplasty balloons without the need to consider pressure equalization between the balloons during bifurcated lesion procedures. Furthermore, due to the independent nature of the valve controls, the valve apparatus can equally well be used in single lesion procedures.

Thus, according to one aspect of the present disclosure there is provided a medical valve apparatus comprising a single female luer inlet, a first side port outlet, a first male luer outlet, a second side port outlet, a second male luer outlet, and first and second valve control mechanisms, the valve control mechanisms being independent and manually operable.

The first valve control mechanism is moveable between a first position where fluid flow between the female luer inlet and both the first side port outlet and the first male luer outlet is blocked, a second position where fluid flow between the female luer inlet and the first side port outlet is blocked, and a third position where fluid flow between the female luer inlet and the first male luer outlet is blocked; and

The second valve control mechanism is moveable between a first position where fluid flow between the female luer inlet and both the second side port outlet and the second male luer outlet is blocked, a second position where fluid flow between the female luer inlet and the second side port outlet is blocked, and a third position where fluid flow between the female luer inlet and the second male luer outlet is blocked.

In some embodiments, one or both of the valve mechanisms comprise a portion external to the apparatus indicating the current position of the valve.

### BRIEF DESCRIPTION OF THE DRAWINGS

Various embodiments of the invention are disclosed in the following detailed description and accompanying drawings.

FIG. 1 illustrates a first isometric view of an example configuration of a medical valve apparatus according to the present disclosure.

FIG. 2 illustrates the same example configuration of a medical valve apparatus according to the present disclosure connected to an indeflator and in the process of inflating two cardiac angioplasty balloons simultaneously.

FIG. 3 illustrates the same example configuration of a medical valve apparatus according to the present disclosure connected to an indeflator and in the process of inflating a single cardiac angioplasty balloon.

FIG. 4 illustrates the same example configuration of a medical valve apparatus according to the present disclosure with the fluid paths between the indeflator and the angioplasty balloon outlined.

Common reference numerals are used throughout the figures and the detailed description to indicate like elements. One skilled in the art will readily recognize that the above figures are examples and that other architectures, modes of operation, orders of operation, and elements/functions can be provided and implemented without departing from the characteristics and features of the invention, as set forth in the claims.

### DETAILED DESCRIPTION AND PREFERRED EMBODIMENT

The following is a detailed description of exemplary embodiments to illustrate the principles of the invention. The embodiments are provided to illustrate aspects of the invention, but the invention is not limited to any embodiment. The scope of the invention encompasses numerous alternatives, modifications and equivalent; it is limited only by the claims.

Numerous specific details are set forth in the following description in order to provide a thorough understanding of the invention. However, the invention may be practiced according to the claims without some or all of these specific details. For the purpose of clarity, technical material that is known in the technical fields related to the invention has not been described in detail so that the invention is not unnecessarily obscured.

The terminology used herein is for the purpose of describing particular embodiments only and is not intended to be limiting of the invention. As used herein, the term “and/or” includes any combinations of one or more of the associated listed items. As used herein, the singular forms “a,” “an,” and “the” are intended to include the plural forms as well as the singular forms, unless the context clearly indicates otherwise. It will be further understood that the terms “comprises” and/or “comprising,” when used in this specification, specify the presence of stated features, steps, operations, elements, and/or components, but do not preclude the presence or addition of one or more other features, steps, operations, elements, components, and/or groups thereof.

The present disclosure relates to a medical valve apparatus formed of a dual three-way stopcock structure sharing a fluid intake but with independent valve control mechanisms.

The inlets and outlets of the valve apparatus are generally in the form of Luer tapers. The Luer taper is a standardized system of small-scale fluid fittings used for making leak-free connections between a male-taper fitting and its mating female part on medical and laboratory instruments, i.e. a stopcock. However, it is also possible for other fluid fittings to be used on the inlets and outlets.

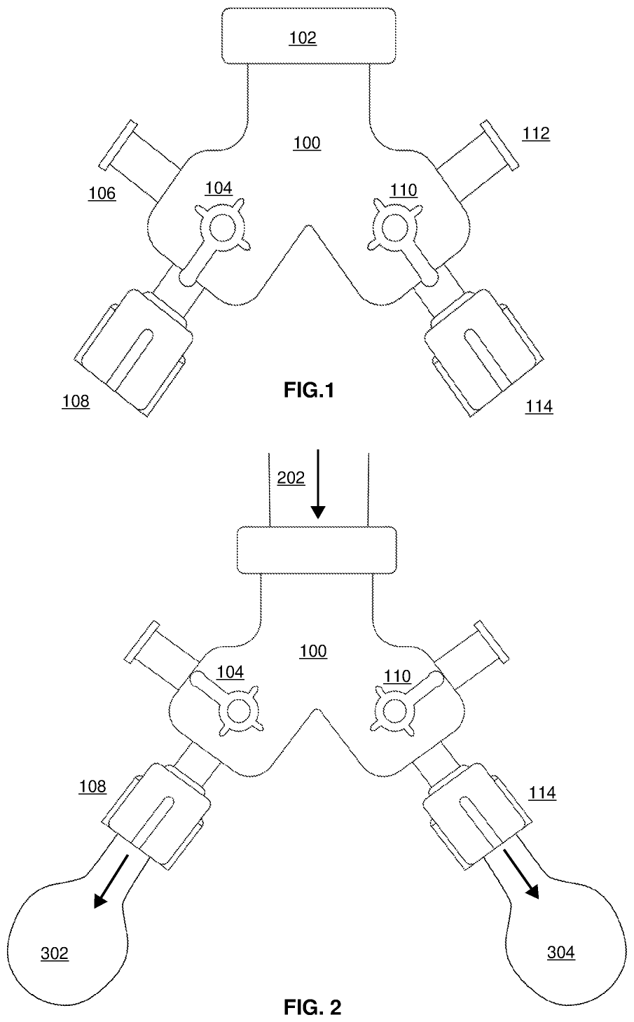

Referring to FIG.1, a first isometric view of an example configuration of a medical valve apparatus 100 according to the present disclosure is shown.

The valve apparatus comprises a single shared female fluid inlet 102 that leads into a branching fluid pathway inside the apparatus 100. One branch of the fluid pathway leads to a first three-way fluid junction between the female fluid inlet 102, a first side port 106, and a first male fluid outlet 108.

The fluid flow through this three-way junction is controlled by a first fluid valve mechanism 104, which is able to move between three different positions to channel fluid between the inlet 102 and the side port 106, the inlet 102 and the outlet 108, or to completely block fluid from the inlet 102 from passing to either the side port 106 or the male outlet 108.

In the present example, the first valve mechanism 104 is shown having a rotatable element disposed on the exterior of the valve apparatus 100 that can be turned through the different positions to control the fluid flow, and which has a directional protrusion that indicates to a user which position the valve is currently in, allowing them to discern the current fluid flow path on the first branch. In the illustrated example, the valve 104 is turned so as to block flow to the male outlet 108, but allow fluid flow between the female inlet 102 and the side port 106.

The second branch of the three-way junction leads to an identical second valve mechanism 110, having its own control over fluid flow between the female inlet 102, a second side port 112, and a second male outlet 114. Similarly, the valve mechanism 110 is currently positioned to block fluid flow to the second male outlet 114.

As explained above, this dual stopcock structure, utilizing a shared female inlet 02 and two independent valve mechanisms 104 and 110 is highly useful for performing cardiac angioplasty on bifurcated arteries with lesions, since two angioplasty balloons can be inflated simultaneously without the consideration of pressure equalization, and by a single operator.

Referring to FIG.2, the same example configuration of the medical valve apparatus 100 is shown with the female inlet 102 connected to an indeflator syringe outlet 202.

The indeflator syringe 202, which would be pre-filled with a saline/contrast solution, is pushing this solution into the valve apparatus 100. Both of the valve mechanism 104 and 110 of the apparatus 100 are currently turned to allow fluid flow only between the female fluid inlet 102 and the first and second male fluid outlets 108 and 114, which is causing first and second angioplasty balloons 302 and 304 to be simultaneously inflated by equal amounts using only one indeflator.

Furthermore, as mentioned above, due to the independent nature of the two valve mechanisms 104 and 110, the valve apparatus 100 can equally well be used for single angioplasty balloon inflations.

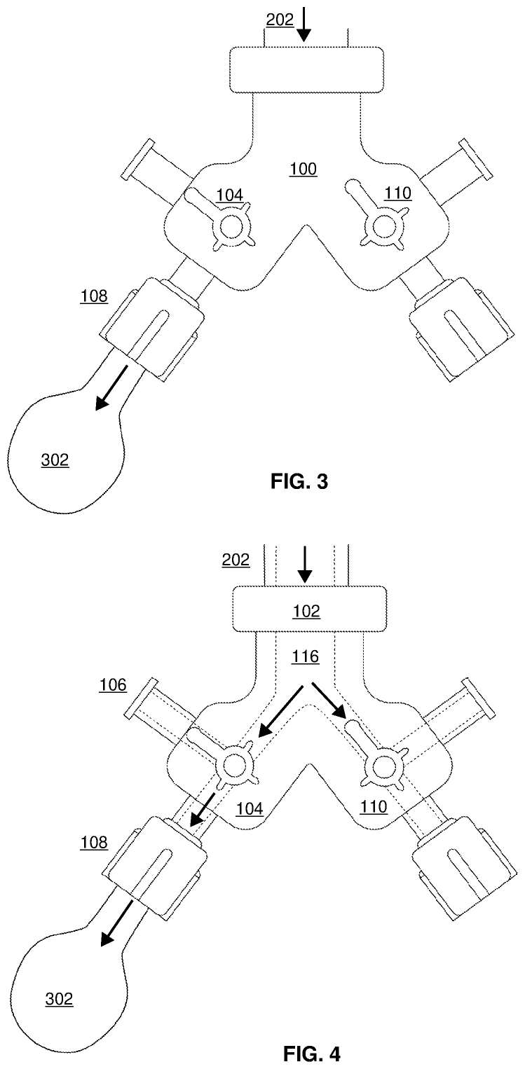

Referring to FIG.3, the same example configuration of the medical valve apparatus 100 is shown with the female fluid inlet 102 connected to the indeflator syringe 202 and in the process of inflating a single cardiac angioplasty balloon 302 which is attached to the first male fluid outlet 108, the fluid flow being allowed through since the first valve mechanism 104 is turned to only block fluid flow to the first side port 106.

In this example, fluid flow to the second branch is completely cut off by the second valve apparatus 110, which is turned to a position so as to block fluid flow to both the second side port and the second male fluid outlet.

The ability to use the valve apparatus 100 for both single and double balloon inflations saves time and expense by avoiding the need to constantly exchange valves.

Finally, referring to FIG.4, the same example configuration of the medical valve apparatus 100 is shown with the internal fluid pathways between the female fluid inlet 102 and the side ports and outlets outlined, including the three-way junction 116. The positions of the valve mechanisms 104 and 110 are as described for FIG.3.

Unless otherwise defined, all terms (including technical terms) used herein have the same meaning as commonly understood by one having ordinary skill in the art to which this invention belongs. It will be further understood that terms, such as those defined in commonly used dictionaries, should be interpreted as having a meaning that is consistent with their meaning in the context of the relevant art and the present disclosure and will not be interpreted in an idealized or overly formal sense unless expressly so defined herein.

The disclosed embodiments are illustrative, not restrictive. While specific configurations of the medical valve apparatus have been described in a specific manner referring to the illustrated embodiments, it is understood that the present invention can be applied to a wide variety of solutions which fit within the scope and spirit of the claims. There are many alternative ways of implementing the invention.

It is to be understood that the embodiments of the invention herein described are merely illustrative of the application of the principles of the invention. Reference herein to details of the illustrated embodiments is not intended to limit the scope of the claims, which themselves recite those features regarded as essential to the invention.

What is claimed is:

1. A medical valve apparatus comprising a single female luer inlet, a first side port outlet, a first male luer outlet, a second side port outlet, a second male luer outlet, and first and second valve control mechanisms, the valve control mechanisms being independent and manually operable;
wherein the first valve control mechanism is moveable between a first position where fluid flow between the female luer inlet and both the first side port outlet and the first male luer outlet is blocked, a second position where fluid flow between the female luer inlet and the first side port outlet is blocked, and a third position where fluid flow between the female luer inlet and the first male luer outlet is blocked; and
wherein the second valve control mechanism is moveable between a first position where fluid flow between the female luer inlet and both the second side port outlet and the second male luer outlet is blocked, a second position where fluid flow between the female luer inlet and the second side port outlet is blocked, and a third position where fluid flow between the female luer inlet and the second male luer outlet is blocked.

2. A medical valve apparatus according to claim 1, wherein one or both of the valve mechanisms comprise a portion external to the apparatus indicating the current position of the valve.
